(19) United States
(12) Patent Application Publication
(10) Pub. No.: US 2005/0015077 A1
(43) Pub. Date: Jan. 20, 2005

(54) Method and apparatus for skin treatment using near infrared laser radiation

(76) Inventors: Yevgeniy Kuklin, Woodland Hills, CA (US); Antoine Garabet, Glendora, CA (US); Nikolay Ivanov, Irkutsk (RU)

Correspondence Address: James E. Brunton, Esquire, Post Office Box 29000, Glendale, CA, 91209-9000, US

(21) Appl. No.: 10/619,573

(22) Filed: Jul. 14, 2003

(51) Int. Cl. A61B 18/20

(52) U.S. Cl. 606/9; 607/89

(57) ABSTRACT

A method and apparatus for the simultaneous removal of superficial epidermal skin cells and unwanted hair using strategically structured laser pulses. The apparatus of the invention includes an optical crystal which, when pumped, will produce uniquely structured pulses of laser light. More particularly, each produced pulse has a first portion of a first duration and a first wavelength and a second portion of a second duration and a second wavelength. In accordance with the method of the invention the first portion of the purse is directed toward a selected target area for a first period of time and in a manner to raise the temperature of the lower portion of the human tissue located within the target area sufficiently to elicit a heating response that will accomplish hair reduction. At the end of the first period of time, the second portion of the pulse of laser light is directed toward the target area for a second period of time in order to raise the temperature of the upper portion of the tissue located within target area sufficiently to elicit a heating response that will rejuvenate the skin located within the target area.

### BACKGROUND OF THE INVENTION

1. Field of the Invention

The present invention relates generally to the treatment of human tissue. More particularly the invention concerns a method and apparatus for the simultaneous treatment of wrinkles in human skin and the removal of unwanted hair using laser pulses

2. Discussion of the Prior Art

In the past, several methods have been suggested which use laser beams to controllably heat the surface layer of skin. This heating of the skin can cause the skin to blister and permit the surface layers of the skin to be scraped away. Similarly, some prior art methods used lasers to vaporize the outer layers of the skin. These prior art processes can have beneficial results but also present substantial risk to the patient. For example, laser treatments can result in pain and accidental burning of the skin, and if not properly carried out can result in bleeding and scarring.

Similarly, several methods have been suggested in the past to accomplish the removal of unwanted hair. Such methods include short-term removal techniques such as shaving and plucking and long-term removal techniques such as electrolysis. Frequently, the prior art methods for permanent or long-term hair removal are painful and often very time consuming. As in the case of skin rejuvenation, various methods have been suggested which use laser beams for hair removal.

With respect to hair removal, it is well understood by those skilled in the art, that human hair comprises two basic parts, the shaft, which is the portion of the hair above the epidermis, and the root, which is the portion of the hair below the surface of the epidermis. Hair color, which is due to the presence of melanin in the hair, is passed into the hair as it grows. It is the presence of melanin that makes it possible to use lasers and other light sources for hair removal with melanin as the target chromophore. The hair tissue, which comprises the hair follicle and the surrounding structure, is selectively heated when the melanin in the hair tissue is exposed to laser pulses. During the typical prior art laser treatment, the hair tissue is thermally damaged so that many of the exposed hairs atrophy and are sloughed from the epidermis.

U.S. Pat. No. 6,383,176 issued to Connors, et al. discloses one approach to the elimination of unwanted hair using laser processes. As discussed in the Connors patent, the early laser work was centered around a wavelength with very high melanin absorption, such as a wavelength of 694 nanometers (nm) generated by a pulsed ruby laser. Long pulse ruby lasers (as opposed to Q-switched ruby lasers) typically have a pulse duration in the 1 millisecond range. Although the wavelength is highly absorbed in melanin, the wavelength selection has significant limitations with darker skin types as the epidermis can blister from the superficial melanin heating.

A common trend in a laser hair removal is a continual shift towards longer wavelengths, which have less melanin absorption. Such approaches allow treatment of patients with a darker range of skin tones. As pointed out in Connors et al, one commercially-available system, sold by Coherent of Santa Clara under the name and style LIGHTSHEER, allows the physician to treat the darkest skin types with minimal risk of post operative blistering. Unfortunately, the high pulse energy diode approach is very expensive, as it requires up to 100 diode bars to achieve the peak powers needed for the desired clinical result.

Another prior art process for skin treatment using a laser is discussed in U.S. Pat. No. 5,817,089 issued to Tankovich, et al. This patent describes a skin treatment process for the removal of superficial epidermal skin cells in the human skin, the reduction or removal of unwanted hair and the mitigation of skin conditions such as acne and seborrhea. The Tankovich, et al. process uses a contaminant having a high absorption at least one wavelength of light which is topically applied to a section of the surface of the skin. Portions of the contaminant are forced to infiltrate into spaces between the superficial epidermal cells, into hair ducts in the skin and into adjacent to sebaceous glands. The skin section is illuminated with short laser pulses at a matching wavelength, so as to impart sufficient energy to the contaminant to cause an explosion in the contaminant. The energy released in the course of the explosion may blow off layers of dead skin cells and destroy the tissue that is responsible for hair growth and sebum production.

Still another prior art process for skin treatment is discussed in U.S. Pat. No. 6,120,497 issued to Anderson et al. This latter patent discloses a method for treating wrinkles in skin, which involves the use of a beam of pulsed, scanned or gated continuous wave laser or incoherent radiation. The method comprises generating a beam of radiation, directing the beam of radiation to a targeted dermal region between 100 microns and 1.2 millimeters below a wrinkle in the skin, and thermally injuring fibroblasts in the targeted dermal region. The beam of radiation has a wavelength of between 1.3 and 1.8 microns. The method may include cooling an area of the skin above the targeted dermal region while partially denaturing the collagen in the targeted dermal region. The method may also include cooling an area of the skin above the targeted dermal region prior to thermally injuring fibroblasts in the targeted dermal region. However, this patent neither discloses nor suggests a method wherein a wavelength is switched during a pulse generation when the pulse duration is less than the thermal relaxation time of the target tissue.

### SUMMARY OF THE INVENTION

It is an object of the present invention to provide a novel method and apparatus for the treatment of wrinkles in the human skin and for the removal of unwanted hair using strategically structured laser pulses.

Another object of the invention is to provide a novel apparatus which will produce pulses of laser light wherein each pulse has a first portion of a first duration and a first wavelength and a second portion of a second duration and a second wavelength.

Another object of the invention is to provide a novel method of skin treatment which uses the apparatus as described in the preceding paragraph in a manner to direct the first portion of the pulse of laser light at a target area for a first duration to raise the temperature of the lower portion of the human tissue located within the target area sufficiently to elicit a heating response that will accomplish hair reduction.

Another object of the invention is to provide a method of skin treatment of the character described in which, subsequent to the first duration, the apparatus of the invention is used to direct the second portion of the pulse of laser light to the target area for a second duration to raise the temperature of the upper portion of the tissue located within target area sufficiently to elicit a heating response that will rejuvenate the skin located within the target area.

Another object of the invention is to provide an apparatus, which is of a simple construction and uses either conventional Nd:YAG or Nd:YAP crystals to generate the pulses of laser light used in carrying out the method of the invention.

Another object of the invention is to provide an apparatus as described in the preceding paragraph, which can be used to generate laser light at a wavelength of approximately 1078 nm and can also be used to generate laser light at approximately 1340 nm.

Another object of the invention is to provide an apparatus as described in the preceding paragraph, which includes means for cooling the target area while simultaneously delivering the pulses of laser light.

Another object of the invention is to provide an apparatus of the class described which is easy and safe to use and one, which can be operated by a laser technician with a minimum amount of training.

By way of summary, one form of the method of the invention for the simultaneous removal of superficial epidermal skin cells and unwanted hair using strategically structured laser pulses makes use of a novel apparatus which includes an optical crystal which, when pumped, will produce uniquely structured pulses of laser light. More particularly, each pulse has a first portion of a first duration and a first wavelength and a second portion of a second duration and a second wavelength. In accordance with a method of the invention, the first portion of the pulse is directed toward a selected target area for a first period of time and in a manner to raise the temperature of the lower portion of the human tissue located within the target area sufficiently to elicit a heating response that will accomplish hair reduction. At the end of the first period of time, the second portion of the pulse of laser light is directed toward to the target area for a second period of time in order to take advantage of the first portion of the pulse of laser light and raise the temperature of the upper portion of the tissue located within target area sufficiently to elicit a heating response that will rejuvenate the skin located within the target area. In one form of the method of the invention, a conventional Nd:YAP crystal is used to generate the pulses of laser light. In an alternate form of the invention a conventional Nd:YAG crystal is used to generate the pulses of laser light.

### BRIEF DESCRIPTION OF THE DRAWINGS

FIG. 1 is a generally diagrammatic view of one form of the apparatus of the invention for accomplishing skin treatment.

FIG. 2 is a generally diagrammatic view of an alternate form of the apparatus for accomplishing skin treatment.

FIG. 3 is a generally diagrammatic view of still another form of the apparatus of the invention.

### DISCUSSION OF THE INVENTION

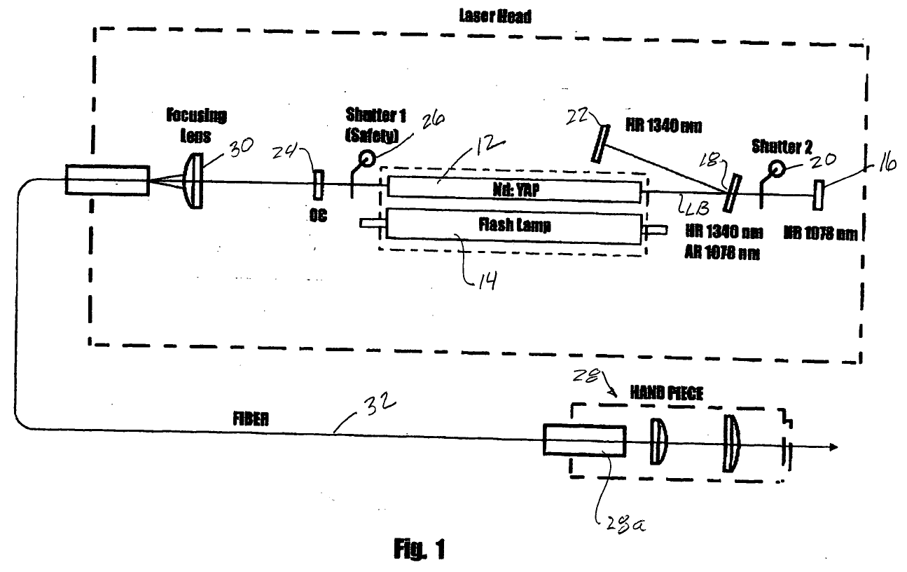

Referring to the drawings and particularly to FIG. 1, one form of the apparatus of the invention for accomplishing skin treatment is there illustrated. As can be seen in FIG. 1, the apparatus here comprises a Nd:YAP rod 12, a pumping light source, such as a flash lamp 14, for radiating pumping light toward the rod 12 and a suitable power supply (not shown) for providing the pumping light source with a suitable input power. As will be discussed in greater detail hereinafter, in carrying out the method of the invention, the laser beam produced by rod 12 is pulsed at a repetition rate of between about 0.1 Hz and about 100 Hz. The pulse duration can range from between about 0.1 millisecond and about 200 millisecond, and the fluence can range between about 1 and about 150 J/CM².

The Nd YAP rod is readily commercially available from various sources, including the German company Impex High-Tech. Similarly, the light source is commercially available from a number of sources, including Heraeus Optics of Buford, Ga.

Provided proximate one end of rod 12 is a conventional high reflectance mirror 16, which exhibits not less than 99% reflectivity at a wavelength of 1078 nm. Disposed within the light beam “LB” produced by rod 12 and located intermediate rod 12 and mirror 16 is a beam turning mirror 18 which is specially coated so as not to reflect light produced at a wavelength of 1078 nm. Positioned between mirror 16 and mirror 18 is a shutter 20 which is used in carrying out the method of the invention to switch wavelengths in a manner presently to be described. With the construction shown, when shutter 20 is open and both resonant conditions exist, the laser will produce light only at the 1078 nm wavelength because this mode is dominant. However, when shutter 20 is closed, the laser will produce light only at the 1340 nm wavelength. Shutter 20 can comprise an electro-optical based switch, a fast mechanical shutter or a rotating mask. In any event, shutter 20 must have a switching time of less than two percent of the total duration of the pulse produced. A shutter suitable for present purposes is commercially available from sources such as Goocha Housego of Somerset, U.K.

Turning mirror 18 exhibits high reflectivity of light at a wavelength of 1340 nm and, as shown in FIG. 1, functions to reflect the light beam “LB” toward the high reflectivity mirror 22 which also comprises a part of the form of the apparatus of the invention shown in FIG. 1.

Also forming a part of the apparatus of the invention is an output coupler 24, which is reflective at wavelengths of 1078 and 1340 nm, and a safety shutter 26 which is disposed between output coupler 24 and rod 12. Safety shutter 26, when closed, functions to safely disable the apparatus. The reflectivity of output coupler 24 is about 37% at wavelengths of 1078 nm and about 62% at wavelengths of 1340 nm and is chosen to achieve maximum lasing efficiency at each wavelength. Disposed between output coupler 24 and a hand piece, generally designated by the numeral 28, is a focusing lens 30 which functions to collimate the light beam into an optical fiber 32 which extends between the lens and the hand piece. Hand piece 28 is of conventional construction and is constructed from components which are readily commercially available. Lens 30, which has a focal length of 25 mm and antireflection coating for both 1078 nm and 1340 nm wavelengths, is available from commercial sources such as Lambda Research Optics located in Costa Mesa, Calif. The mirrors 16 and 22 are also commercially available from Lambda Research Optics.

In contemplating the use of lasers for treatment of human tissue, several factors must be considered. Initially, it should be understood that the thermal effect of lasers on biological tissue is a complex process resulting from three distinct phenomena, namely the conversion of light to heat, the transfer of heat to the tissue and the reaction of the tissue to the heat.

The known factors comprise the parameters of the laser, such as wavelength, power, time and mode of emission, beam profile and spot size and the characteristics of the tissue being treated.

Additionally, it should be understood that the conversion of light absorbed to heat produces “primary” heat that is transferred through the tissue by the mechanism of conduction. In this regard, it is to be noted that the influence of blood circulation, that is the transfer of heat by convection, is negligible. Conduction may be considered to be like a transfer of energy by interaction with tissue particles. This transfer occurs randomly between the more and the less energetic particles and results in a “secondary” heated volume that comprises an important part of the process of thermal injury of tissue, which is the final result of thermal action on tissue. The kinetics of this transformation depend on the temperature of the tissue, the time of heating and on the susceptibility of the tissue to thermal damage.

Due to the optical properties of tissue, the penetration depth and the heat distribution are different for different wavelengths. As will be discussed in greater detail hereinafter, by switching wavelength during pulse generation, when pulse duration is less than the thermal relaxation time of tissue, will produce variable heat distribution depending on time and energy of each portion of delivered pulse.

In carrying out the methods of the invention, each portion of the delivered pulse produces a maximum of “primary” thermal effect at a certain depth. This thermal effect depends on the power and duration of time of each portion of the pulse. Combining the first and second portions of the pulse will produce a “secondary” thermal effect with a predetermined heat distribution within the tissue. By controllably varying these thermal effects, one can select the most effective profile of thermal injury for a particular type of skin and for a particular purpose.

With the foregoing in mind, various experiments have been conducted which demonstrate that laser radiation at a wavelength of approximately 1078 nm is useful in hair reduction processes, but is not particularly useful in rejuvenation processes. On the other hand, these same experiments have shown that laser radiation at a wavelength of approximately 1340 nm provides better results in skin rejuvenation processes, but is of little value in hair reduction.

Studies of laser irradiated tissue have also demonstrated that radiation at the 1340 nm wavelength penetrates to a lesser depth and locally drives the temperature higher than does radiation at the 1078 nm wavelength. On the other hand, the studies have shown that the time for heating the skin as well as the relaxation time of the skin for radiation at the 1078 nm wavelength is much longer than for radiation at the 1340 nm wavelength.

Experimentation has further determined that simultaneous radiation of the tissue at both the 1340 nm and the 1078 nm wavelengths achieves very limited results. It has also been determined that similar results occur if the tissue is separately radiated at the 1340 nm and 1078 nm wavelengths. However, as will be better understood from the discussion which follows, using the uniquely structured pulses of light, for example at 1340 nm and the 1078 nm wavelengths produces novel and unexpected beneficial results.

More particularly, in accordance with one form of the method of the present invention, effective hair reduction can be accomplished by irradiating the tissue with laser light at the 1078 nm wavelength, while at the same time cooling the epidermis. Since it takes a significant length of time for the volume of the tissue heated by the 1078 nm wavelength radiation to relax, it is possible, in accordance with one form of the method of the present invention, to irradiate the tissue during this relaxation period with the laser light at the 1340 nm wavelength in a manner to preserve the earlier tissue energization that resulted from the laser light at the 1078 nm wavelength. This second irradiation step raises the temperature of the upper dermis to a desired level, while safely interacting with the epidermis. Additionally, because of the time delay in irradiating the tissue with the 1340 nm wavelength radiation, the epidermis, which was heated by the 1078 nm wavelength radiation, is permitted to controllably cool down.

By way of example, in the accomplishment of one form of the method of the present invention, the tissue is first irradiated by a first portion of a laser pulse at the 1078 nm wavelength for a duration of time of on the order of the time for relaxation of the hair follicle, which is greater than the time of relaxation of the epidermis. In this way, the temperature of the hair follicle can be controllably raised to a destructive level. At the time, when the light at a wavelength of 1340 nm is applied, the epidermis is already relaxed, since it is cooled by external cooling and also because the time of exposure to 1078 nm light is greater than the time of relaxation of the epidermis. Additionally, since the time period for relaxation of the dermis following exposure to the light at the 1078 nm wavelength is considerably longer than the time of relaxation of the dermis following exposure to the light at a wavelength of 1340 nm, the tissue still embodies the heat resulting from the exposure of the tissue to the light at a wavelength of 1078 nm. Therefore, the radiation at the 1340 nm wavelength, which is used to destroy the tissue in the upper dermis region in order to accomplish rejuvenation of the tissue, can be applied in lesser energy densities, thereby permitting safer interaction with the epidermis.

In accordance with the methods of the present invention, as described in the preceding paragraphs, by applying to the tissue a delayed pulse of radiation at about the 1340 nm wavelength immediately following the irradiation of the tissue with radiation at about the 1078 nm wavelength, it is possible to achieve both hair reduction and rejuvenation simultaneously, while at the same time safely protecting epidermis. Clearly such results are not achievable by using both wavelengths of radiation without an appropriate time delay.

By way of example, one form of the tissue treatment method of the present invention for treating, through the use of a Nd:YAP crystal 12, tissue located at a selected the target area and having an upper and lower portion, comprises the steps of:
1. Pumping crystal 12 through use of light source 14 (FIG. 1) to generate a pulse of laser light having a first portion having a first wavelength of about 1078 nm and a fluence of between about 10 and about 100 J/cm2 and a second portion having a second wavelength of about the 1340 nm and a fluence of between about 1 and about 50 J/cm2;
2. While cooling the selected target area using cooling means 28a, directing the first portion of the pulse of laser light to a target area for a first duration of time of about 10 and 20 microseconds in order to raise the temperature of the lower portion of the human tissue within the target area to an extent that is sufficient to elicit a heating response that will accomplish hair reduction within the target area; and
3. Following the first duration of time, directing the second portion of the pulse of laser light to the target area for a second duration of time of between about 3 and 6 milliseconds in order to raise the temperature of the upper portion of the human tissue within the target area to an extent that is sufficient to elicit a heating response that will accomplish skin rejuvenation within the target area.

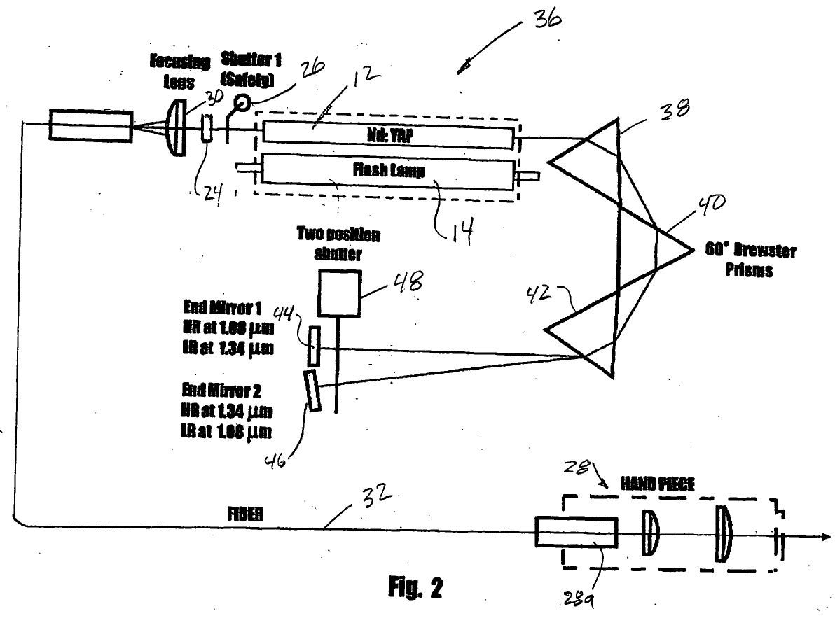

Turning to FIG. 2, an alternate form of the apparatus of the present invention is there illustrated and a generally identified by the numeral 36. This alternate form of the apparatus is similar in many respects to that shown in FIG. 1 and like numerals are used in FIG. 2 to identify like components. The primary difference between this latest form of the invention and the earlier described form of the invention resides in the fact that a plurality of cooperating Brewster prisms 38, 40 and 42 are used to a separate the wavelengths. The Brewster prisms are spaced apart from the first end of a Nd:YAP crystal 12 in the manner shown in FIG. 2.

Positioned remotely from the prisms are first and second end mirrors 44 and 46 respectively. Disposed between mirrors 44 and 46 and the Brewster prisms is a two-position shutter 48.

Also forming a part of the apparatus of the invention is an output coupler 24, which is reflective at wavelengths of 1078 and 1340 nm, and a safety shutter 26 which is disposed between output coupler 24 and rod 12. These components are of the same construction and operate in the same manner as those discussed in connection with the embodiment of FIG. 1. As before, when closed, safety shutter 26, functions to safely disable the apparatus. Disposed between output coupler 24 and a hand piece, generally designated by the numeral 28, is a focusing lens 30 which functions to collimate the light beam into an optical fiber 32 which extends between the lens and the hand piece. These last identified components are also identical in construction and operation to those previously discussed herein.

In operation of the apparatus shown in FIG. 2, depending upon the position of the shutter 48, either end mirror 44 or end mirror 46, which are included within the resonant cavity, determine the generated wavelength because of their selective reflection of light at the 1078 nm wavelength and at the 1340 nm wavelength. It is to be understood that a rotating masked or other like mechanical component could be used in lieu of shutter 48.

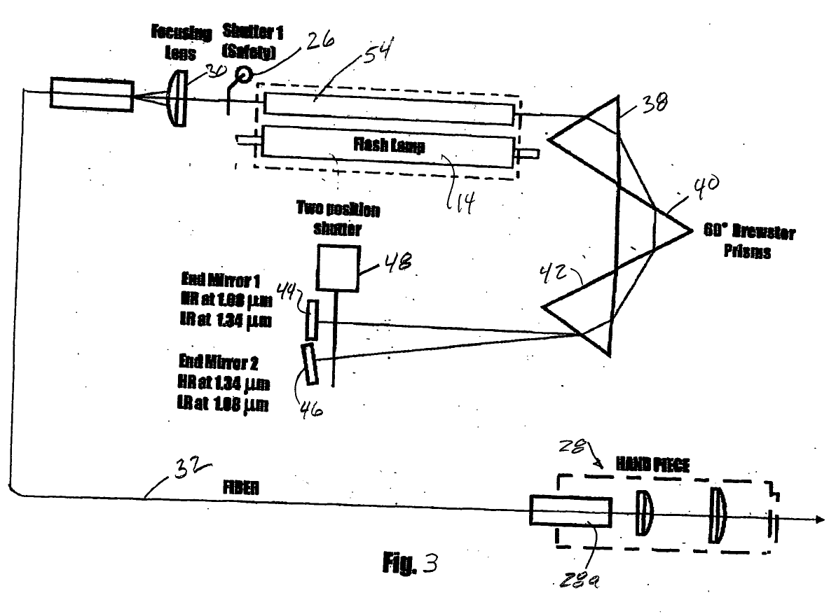

Referring lastly to FIG. 3, still another form of the apparatus of the present invention is there illustrated and generally identified by the numeral 52. This latest form of the invention is similar in construction in operation to the apparatus shown in FIG. 2 and like numerals are used in FIG. 3 to identify like components. However, as shown in FIG. 3, rather than using a Nd:YAP crystal rod to produce the laser light pulses, a Nd:YAG crystal rod 54 is used.

By way of further example, another form of the tissue treatment method of the invention for treating a selected target area makes use of the alternate form of the apparatus of the invention shown in FIG. 3 and a Nd:YAG crystal 54 is used instead of a Nd:YAP crystal 12 to produce the laser light. This alternative form of the method of the invention is similar in many respects to that described in the preceding paragraphs and comprises the steps of:
1. Pumping crystal 54 through use of a light source 14 to generate a pulse of laser light having a first portion having a first wavelength of about 1064 nm and a second portion having a second wavelength of about the 1310 nm;
2. While cooling the selected target area, directing the first portion of the pulse of laser light to a target area for a first duration of time of about 10 and 20 microseconds in order to raise the temperature of the lower portion of the human tissue within the target area to an extent that is sufficient to elicit a heating response that will accomplish hair reduction within the target area; and
3. Following the first duration of time, directing the second portion of the pulse of laser light to the target area for a second duration of time of between about 3 and 6 milliseconds in order to raise the temperature of the upper portion of the human tissue within the target area to an extent that is sufficient to elicit a heating response that will accomplish skin rejuvenation within the target area.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A method for treating human tissue having an upper portion and a lower portion, comprising the steps of:
(a) using an optical crystal, generating a pulse of laser light, said pulse having a first portion of a first duration and a first wavelength and a second portion of a second duration and a second wavelength;
(b) directing said first portion of said pulse of laser light toward a target area for a first duration of time sufficient to raise the temperature of the lower portion of the human tissue to an extent sufficient to elicit a heating response that will accomplish hair reduction; and
(c) following said first duration, directing said second portion of said pulse of laser light toward said target area for a second duration of time sufficient to raise the temperature of the upper portion to an extent sufficient to elicit a heating response that will accomplish skin rejuvenation.

2. The method as defined in claim 1 in which said optical crystal comprises a Nd:YAP crystal and in which said first wavelength of laser light is approximately 1078 nm and in which said second wavelength of laser light is approximately 1340 nm.

3. The method as defined in claim 1 in which said optical crystal comprises a Nd:YAG crystal and in which said first wavelength of light is approximately 1064 nm and in which said second wavelength of laser light is approximately 1310 nm.

4. The method as defined in claim 1 including the further step of determining the period of relaxation of the human tissue following the exposure thereof to said first portion of said pulse of laser light and within said period, directing said second portion of said pulse of lighting toward said target area.

5. The method as defined in claim 1 in which said optical crystal comprises a Nd:YAP rod that produces said first portion of said pulse of light at a wavelength of about 1078 nm with fluence of between about 10 and about 100 J/CM² and produces said second portion of said pulse of light at a wavelength of 1340 nm with a fluence of between 1 and about 50 J/CM².

6. A method for treating human tissue having an upper dermis and the lower portion, comprising the steps of:
(a) using a Ng: YAP crystal, generating a pulse of laser light, said pulse having a first portion of a first duration and a wavelength of approximately 1078 nm a second portion of a second duration and a wavelength of approximately 1340 nm;
(b) directing the first portion of said pulse of laser light to a target area for a first duration of time sufficient to raise the temperature of the lower portion of the human tissue to an extent sufficient to elicit a heating response that will accomplish hair reduction; and
(c) following said first duration, directing the second portion of said pulse of laser light to said target area for a second duration of time sufficient to raise the temperature of the upper dermis to an extent sufficient to elicit a heating response that will rejuvenate the skin within said target area.

7. The method as defined in claim 6 including the further step of determining the period of relaxation of the human tissue following the exposure thereof to said first portion of said pulse of laser light and within said period, directing said second portion of said pulse of light toward said target area

8. The method as defined in claim 6 in which said first portion of said pulse is produced with a fluence of between about 35 and 50 J/CM² for a duration of between about 10 and 20 milliseconds.

9. The method as defined in claim 6 in which said second portion of said pulse is produced with a fluence of between about 3 and 6 J/CM² for a duration of between about 3 and 6 milliseconds.

10. The method as defined in claim 6, further including the step of cooling the target area during said first duration of time.

11. An apparatus for use in treating the human skin comprising:
(a) a optical crystal having a first end and a second end;
(b) a pumping light source for irradiating pumping light on said optical crystal;
(c) a first high reflectivity mirror spaced apart from said first end of said optical crystal;
(d) a shutter disposed between said first high reflectivity mirror and said optical crystal;
(e) a second, high reflectivity mirror disposed between said shutter and said optical crystal;
(f) a third high reflectivity mirror spaced apart from said second, rotatable high reflectivity mirror and operably associated therewith;
(g) a focusing lens spaced apart from said second end of said optical crystal;
(h) an output coupler disposed between said focusing lens and said second end of said optical crystal; and
(i) a hand piece spaced apart from and operably associated with said focusing lens.

12. The apparatus as defined in claim 10 in which said optical crystal comprises a Nd:YAP crystal.

13. The apparatus as defined in claim 10 in which said optical crystal comprises a Nd:YAG crystal.

14. The apparatus as defined in claim 10 further including an optical fiber disposed between said focusing lens and said hand piece.

15. The apparatus as defined in claim 10 in which said hand piece includes cooling means for cooling the human skin.

16. An apparatus for use in treating the human skin comprising:
(a) a optical crystal having a first end and a second end;
(b) a pumping light source for irradiating pumping light on said optical crystal;
(c) a first high reflectivity mirror spaced apart from said first end of said optical crystal;
(d) a second high reflectivity mirror spaced apart from said first end of said optical crystal;
(e) a shutter disposed between said first and second high reflectivity mirrors and said optical crystal;
(f) a plurality of prisms disposed between said shutter and said optical crystal;
(g) a focusing lens spaced apart from said second end of said optical crystal;
(h) an output coupler disposed between said focusing lens and said second end of said optical crystal; and
(i) a hand piece spaced apart from and operably associated with said focusing lens.

17. The apparatus as defined in claim 16 in which said shutter comprises a two piston shutter.

18. The apparatus as defined in claim 16 in which said optical crystal comprises a Nd:YAP crystal.

19. The apparatus as defined in claim 16 further including an optical fiber disposed between said focusing lens and said hand piece.

20. The apparatus as defined in claim 16 in which said hand piece includes cooling means for cooling the human skin.
